(19) United States
(12) Patent Application Publication
(10) Pub. No.: US 2025/0205512 A1
(43) Pub. Date: Jun. 26, 2025

(54) PERSONALIZED EYE PROTECTION BLOCK FOR PROTECTING THE CORNEA AND LENS IN TUMORS OCCURRING IN AND AROUND THE EYELID

(71) Applicant: ATATURK UNIVERSITESI REKTÖRLÜGU BILIMSEL ARASTIRMA PROJELERI ( BAP ) KOORDINASYON BIRIMI, Erzurum (TR)

(72) Inventors: KORKMAZ SERIFOGLU, Erzurum (TR); YILMAZ SAHIN, Erzurum (TR)

(73) Assignee: ATATURK UNIVERSITESI REKTÖRLUGU BILIMSEL ARASTIRMA PROJELERI ( BAP ) KOORDINASYON BIRIMI, Erzurum (TR)

(21) Appl. No.: 18/692,845

(22) PCT Filed: Sep. 11, 2022
(86) PCT No.: PCT/TR2023/050938

§ 371 Date: Mar. 18, 2024

(30) Foreign Application Priority Data
Sep. 19, 2022 (TR) 2022/014397

(51) Int. Cl. A61N 5/10; A61F 9/00

(52) U.S. Cl. CPC A61N 5/10; A61F 9/00; A61N 2005/1094

(57) ABSTRACT

The present invention relates to a personalized eye protection block (1) for protecting the cornea and lens in tumors occurring in and around the eyelid so as to minimize the radiation dose received by the cornea and lens during radiotherapy in tumors occurring in and around the eyelid in radiotherapy.

### FIELD OF THE INVENTION

The present invention relates to a personalized eye protection block for protecting the cornea and lens in tumors occurring in and around the eyelid so as to minimize the radiation dose received by the cornea and lens during radiotherapy in tumors occurring in and around the eyelid in radiotherapy.

### STATE OF THE ART

In the state of the art, radiation therapy is applied to tumors or cancers found in the eyelid and around the eye for various reasons. However, these practices have some side effects.

As it is known, when radiotherapy (radiation therapy) is applied to tumors or cancers in the eyelid and around the eye, the lens and cornea of the eye are damaged by receiving too much radiation (over the maximum permissible tolerance dose).

In order to prevent the related damage, it is not possible to talk about a specifically designed part or structure. There is clearly a need for improvement in this context.

Although scalpels with eye protection function and various equipment has been put forward for eye tumor operation with the document numbered CN210749812U on the subject, it has been determined that the demand has still not been met and improvements should be made.

### DEFINITION OF THE INVENTION

Said invention eliminates the disadvantages described in the state of the art and fulfils the needs.

The present invention relates to a personalized eye protection block for protecting the cornea and lens in tumors occurring in and around the eyelid so as to minimize the radiation dose received by the cornea and lens during radiotherapy in tumors occurring in and around the eyelid in radiotherapy.

Said invention reduced the radiation dose to the cornea and lens to the maximum permissible tolerance dose level during radiation treatment of tumors on the eyelid or in the eye neighborhood, thereby protecting the cornea and lens and thus preventing damage. This solution, on the other hand, was realized with the lead alloy block made specifically for the person.

The major advantage of the present invention is that it is a model that can be used reliably in radiotherapy. Since it is very difficult to treat tumors in the eyelid and its contour, especially by protecting the cornea and lens. The radiation dose to the lens and cornea is minimized and the radiation dose is received within tolerance limits, thus protecting the eye with the help of this personalized eye block model.

### REFERENCE LIST

1. Eye protection block
2. Patient's eye width
3. Patient's eye size
4. Patient's eye depth

### BRIEF DESCRIPTION OF DRAWINGS

FIG. 1: Representative top view of the present invention

FIG. 2: Side representative view of the present invention

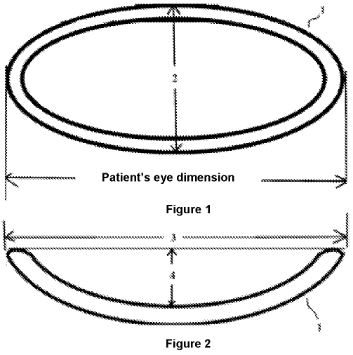

### DESCRIPTION OF THE INVENTION

Personalized eye protection block (1) for protecting the cornea and lens in tumors occurring in and around the eyelid, characterized in that; it is a product which is made of alloy ratios as 50% bismuth, 26.7% lead, 13.3% tin and 10% cadmium, has a melting temperature of around 70° C. and has an acrylic outer coating.

In the production of a personalized eye protection block (1) for protecting the cornea and lens in tumors occurring in and around the eyelid;

a) Since the use of pure lead for the product model would be very difficult in terms of processing the product, Bismuth, Cadmium, tin and Lead were mixed in certain proportions (50% bismuth, 26.7% lead, 13.3% tin and 10% cadmium) for easy processing. In this case, it was made easy to process and the melting temperature was reduced to 70° C.
b) A mold suitable for the person's eye structure (eye width (2), eye size (3), eye depth (4) (concavity) etc.) was formed. A reverse mold was created using this mold. Bismuth, Cadmium, Lead alloy with a thickness of 3 mm was melted and poured into this mold.
c) The mixture was kept at room temperature to cool and harden the same.
d) The unwanted particles on this protection model were thoroughly cleaned and smoothed with zero sandpaper.
e) The eye protection model was covered with a very thin layer of acrylic (dental jaw prosthesis material) against the risk of tissue damage and infection.
f) In order to determine the radiation permeability of this eye protection model, which was made as detailed in Table 1, measurements were taken to determine how much absorption was achieved by using real beam on the device to be used.
g) As can be seen from the measurement results in Table 1, this model absorbs most of the primary beam in the measurement results at 3 different energy types. The measurements showed that it absorbed 100% of the primary beam at an energy of 6 MeV (Million electron Volts), which is the energy type frequently preferred especially in eyelid tumors. Thus, it was determined that, thanks to this eye block, the entire eye received a radiation dose much lower than the maximum permissible dose for the eye specified in the literature.
h) When the results are evaluated; this model shows that the entire eye receives a radiation dose far below the tolerance values in the treatment of tumors in the eyelid and eye contour.

TABLE 1
Permeability coefficients of block protections

| | |
|---|---|
| With focusing block, | 0.80 nCl |
| No eye protection | |
| 06 MeV | Reduces the primary beam by 100% |
| With focusing block, | 0.00 nCl |
| with eye protection | |
| With focusing block, | 1.08 nCl |
| No eye protection | |
| 09 MeV | Reduces the primary beam by 87% |
| With focusing block, | 0.14 nCl |
| with eye protection | |
| With focusing block, | 1.25 nCl |
| No eye protection | |
| 12 MeV | Reduces the primary beam by 49.6% |
| With focusing block, | 0.63 nCl |
| with eye protection | |

1. Personalized eye protection block (1) for protecting the cornea and lens in tumors occurring in and around the eyelid, characterized in that; it is a product which is made of alloy ratios as 50% bismuth, 26.7% lead, 13.3% tin and 10% cadmium, has a melting temperature of around 70° C. and has an acrylic outer coating.

2. Production of a personalized eye protection block (1) for protecting the cornea and lens in tumors occurring in and around the eyelid, characterized in that, it comprises the following process steps;
a) mixing of bismuth, cadmium, tin and lead in certain proportions (50% bismuth, 26.7% lead, 13.3% tin and 10% cadmium) and in this case, making easy to process and reducing the melting temperature to 70 C,
b) making a mold suitable for the person's eye structure (eye width (2), eye size (3), eye depth (4) (concavity)), creating a reverse mold using this mold, pouring bismuth, cadmium, lead alloy, which is formed 3 mm thick, into this mold,
c) keeping the mixture at room temperature so as to cool and harden the same.
d) cleaning the unwanted particles on this protection model thoroughly and smoothing the same with zero sandpaper,
e) covering the eye protection model with a very thin layer of acrylic (dental jaw prosthesis material) against the risk of tissue damage and infection.
